(19) United States
(12) Patent Application Publication
(10) Pub. No.: US 2011/0184329 A1
(43) Pub. Date: Jul. 28, 2011

(54) Composite Arterial-Venous Shunt System

(76) Inventors: Valentin Kramer, Feldkirchen-Westerham (DE); Beda Bruce-Gerald Ruefer, Bozeman, MT (US)

(21) Appl. No.: 12/692,375

(22) Filed: Jan. 22, 2010

(51) Int. Cl. A61M 5/00; A61B 17/11

(52) U.S. Cl. 604/8; 606/153

(57) ABSTRACT

A novel composite arterial-venous (AV) shunt system is disclosed that resolves many known and persistent clinical problems associated with traditional synthetic AV shunts. The system comprises fluid-dynamically optimized non-porous anastomotic connectors with a non-porous self-sealing tubular shunt, and resolves clinical problems of leakage, infection, clotting due to turbulence, and cellular in-growth that can lead to stenosis.

### BACKGROUND OF THE INVENTION

The present invention relates generally to vascular access required for hemodialysis. Vascular access is normally obtained by direct puncture with a needle or cannula into a patient's veins. Because of the repetitive requirement for vascular access by patients requiring hemodialysis, multiple needle punctures into the patient's veins result in permanent damage and scaring to the veins. When suitable veins are no longer available because of the damage and scaring, needle puncture into a fistula or shunt that has been surgically created for access is preferred.

The current state-of-the-art devices commonly used to make shunts for vascular access consist of expanded porous PTFE tubes (grafts) placed as arterial-venous shunts. Although these devices have been used for many years with limited success, numerous clinical problems continue to be associated with their use. These problems include, but are not limited to, leakage, mechanical failure, clotting, infection, steal syndrome, and anastomotic stenosis. All of these clinical problems summed up lead to early failure of expanded porous PTFE grafts used as shunts and poor clinical performance.

### SUMMARY OF THE INVENTION

Embodiments of the invention include a system for diverting blood from an artery into a vein. The system may include a first substantially non-porous PTFE member. This member has a tubular portion sized for insertion into an artery, and an extraction conduit for diverting blood into a shunt. The system also has a second substantially non-porous PTFE member having a tubular portion sized for insertion into a vein along with an induction conduit for receiving blood from the shunt. The shunt is connectable between said extraction and induction conduits, and has an inner member and an outer member. The inner and outer members are coaxial, and one of the inner and outer members is tubular and made of an elastic substance. The other of the inner and outer members is a tight coil constructed to allow for the passage of a needle into it. Then the coil springs back into place to close off an aperture created by the needle.

Other embodiments of the invention include an arterial-venous shunt system that includes an arterial anastomotic connector for diverting blood into a shunt; a venous anastomotic connector for returning the blood from the shunt to the body. The arterial and venous connectors, in embodiments, are constructed of a biologically-compatible substantially nonporous material, e.g., PTFE.

In some embodiments the arterial anastomotic connector includes an insertable tubular portion and a branched-off diverting side conduit. The side conduit branches off from the tubular portion and extends so that blood is diverted at an angle which is acute relative to the direction of blood flow in the artery. In some embodiments the angle is about 20 degrees.

The venous anastomotic connector (in embodiments) includes an insertable tubular portion and a receiving side conduit. The side conduit extends out at an angle such that blood will be received into a vein at an angle which is acute relative to the direction of blood flow. Like with the other connector, the angle, in embodiments, is about 20 degrees.

Embodiments of the shunt used can also be non-porous and tubular. The shunt can also have self-sealing features. These features might include an outer non-porous elastic tube (made of Silicone in embodiments) and an inner tightly-wound coil (a stainless steel spring in embodiments).

In some embodiments the arterial anastomotic connector includes an insertable tubular portion having an inside diameter at an upstream that is substantially equal to an inside diameter of a selected artery. Similarly, embodiments for the venous anastomotic connector can include an insertable tubular portion having an inside diameter at an upstream that is substantially equal to an inside diameter of a selected vein.

In particular embodiments more specific to the shunt construction, the system for diverting a bodily fluid can include means to divert a supply of fluid from a first bodily vessel into a second bodily vessel through a shunt, the shunt having a coaxial self-sealing arrangement, the coaxial self-sealing arrangement having an inner member and an outer member; one of the inner and outer members being tubular and made of an elastic substance; and the other of the inner and outer members being a tight coil, the coil being constructed to allow for the passage of a needle there through, and then spring back into place to close off an aperture created by the needle. In embodiments the coil (e.g., a stainless steel spring) is located coaxially inside the tubular elastic member. The tubular elastic member may be constructed of silicone.

Methods are also presented. For example, some embodiments are directed to a process for diverting blood from an artery into a vein. These methods involve (i) surgically inserting a first substantially non-porous tubular PTFE member into an artery, the first PTFE member having an extraction conduit for diverting blood into a shunt; (ii) surgically inserting a second substantially non-porous tubular PTFE member into a vein, the second PTFE member having an induction conduit; for receiving blood from the shunt; and (iii) connecting the shunt between the extraction conduit and the induction conduit. The shunt used in these processes can be formed by including a tightly-wound coil coaxially inside an elastic outer tube. Both the extraction and induction conduits can be tapered away from where they join the tubular members to match up with the larger inside diameters of the shunt.

### BRIEF DESCRIPTION OF DRAWINGS

FIG. 1 is a two dimensional drawing showing the anastomotic connectors and the tubular shunt that make up the composite AV shunt system.

FIG. 2 is a two dimensional cross-sectional drawing showing the anastomotic connector.

FIG. 3 is a three-dimensional drawing showing the anastomotic connector being placed into a typical artery or vein.

FIG. 4 is a three-dimensional drawing showing the anastomotic connector secured into a typical artery or vein with sutures.

FIG. 5 is a two-dimensional longitudinal cross-sectional drawing of a portion of the tubular shunt showing the outer silicone tube and the inner stainless steel spring or coil.

FIG. 6 is a two-dimensional transverse cross-sectional view of the tubular shunt taken from Section 6-6 shown in FIG. 5 showing the outer non-porous silicone tube and the inner stainless steel spring or coil.

FIG. 7 is a three-dimensional schematic of the shunt implanted in situ within the human arm.

FIG. 8 is a close up three-dimensional drawing of the tubular shunt showing the opening of the silicone tube and stainless steel spring or coil as a needle is inserted through the tubular shunt wall.

FIG. 9 is a close up three dimensional drawing of the tubular shunt showing the closure of the silicone tube and stainless steel spring coils after a needle is removed from the tubular shunt wall.

### DETAILED DESCRIPTION

Generally, the disclosed systems and methods, in embodiments, involve an assembly of parts for a novel arterial-venous shunt system and processes for using these components. In embodiments, non-porous biocompatible connectors serve as anastomotic devices joining an artery or vein to a non-porous tubular shunt. The shunt is non-porous, and attaches to the connectors, which are also nonporous. The preferred material for the anastomotic connectors, in embodiments, is high density, nonporous polytetrafluoroethylene or PTFE. The preferred material for the tubular shunt, in embodiments, is silicone tubing containing a continuous length of stainless steel coil or spring.

As will be evident to one skilled in the art, the system components may be constructed in a variety of shapes and sizes as specific clinical needs require without taking away the novelty of the invention. It should also be noted that the use of the term “shunt” as it appears throughout this document is not to be interpreted narrowly (e.g., as being limited to any particular kind of device) but instead could be any means of passage between two separate body channels.

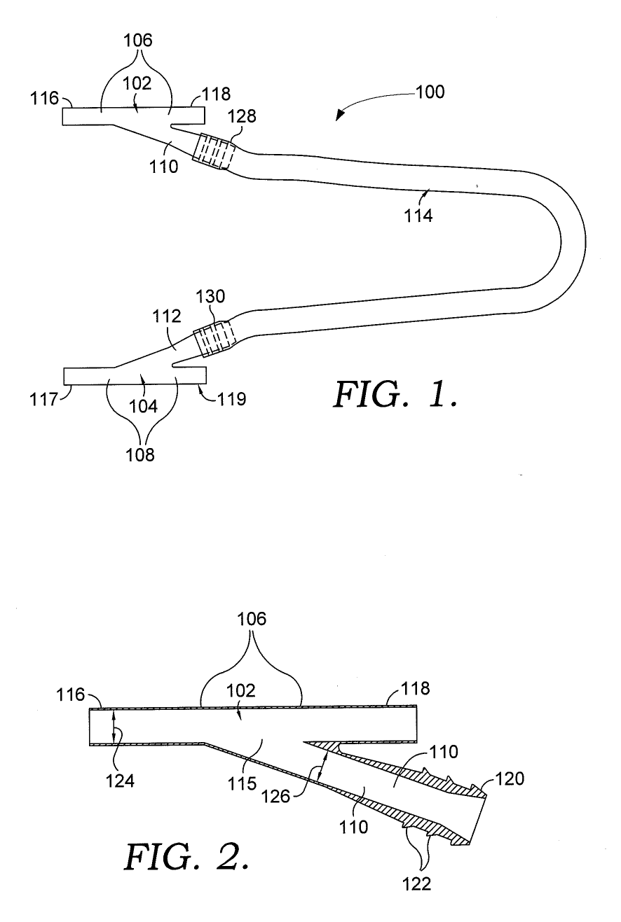

An embodiment 100 of the system can be seen in FIGS. 1-9. FIG. 1 shows a two-dimensional overall view of the composite AV shunt system 100. System 100 includes two anastomotic connectors, 102 and 104, respectively. The details regarding each of connectors 102 and 104 can best be seen in the cross section of FIG. 2 (which is of connector 102, but substantially representative of both). Each connector has a straight base-tube portion—numbered 106 for connector 102, and 108 for connector 104. Straight section 106 enables connector 102 to be inserted into the patient's artery, and section 108 enables connector 108 to be inserted into the patient's vein when the system is used for conducting hemodialysis. Connector 102, also includes an outwardly angled side tube 110 that will be used to secure one end of the shunt 114. Similarly, connector 104 has an outwardly angled side tube 112 which receives the other end of shunt 114. Tubular shunt 114 will be tapped in order to conduct hemodialysis procedure after it is connected in surgery.

FIG. 2, which is a cross section of connector 102, but also accurately reflects the geometries of identical (or substantially identical) anastomotic connector 104. The anatomotic connectors can include tubular features. The straight base-tube portion 106 has a first end 116 and a second end 118. Ends 116 and 118 will be inserted into the patient's artery (or vein) and then sutured in leaving angled portion 110 sticking out. If connector 102 is used to tap into the artery in the procedure, end 116 would be inserted into the upstream direction of the artery to receive blood under pressure. End 118 will thus be inserted into the downstream direction to maintain blood flow downstream (although at a reduced level). Angled out side conduit 110 will be used to draw blood for use in hemodialysis.

The connector inserted into the vein (connector 104) will have the opposite orientation as just discussed. More specifically, a first end 117 of connector 104 will be inserted in the vein in the downstream direction of blood flow and a second end 119 will be directed upstream. Angled side conduit 112 will be used to receive blood (and potentially other introduced substances depending on the procedure) from shunt 114 back into the patient's cardiovascular system.

The connectors will be received snugly into the patient's vein or artery such that the vein or artery can be closed following insertion of the straight base-tube portion of the connector.

Although the cross-section of FIG. 2 is representative of the geometries of each of connectors 102 and 104, the precise diameters, in the preferred embodiments are adapted to optimize performance. More specifically, the inside diameter 124 at end 116 of connector 102 is approximately the same as the inside diameter of the artery into which the straight base-tube 106 is being inserted. Similarly, the inside diameter (not shown) at end 117 is made to be about the same as the inside diameter of the vein into which is installed. This close approximation of the inside diameters of the straight base-tubes and respective vein or artery ensures that blood flow is not impeded and turbulence is minimized. The matching of diameters to the artery and vein upon installation is facilitated by the minimal wall thickness of straight base-tubes 106 and 108.

With respect to connector 102, the bore size 126 of side tube 110 is approximately the same as the straight base-tube at the point of juncture 115 of the two tubes (see FIG. 2). The internal diameter of the side tube 110 gradually tapers outward from the juncture point 115 to the distal end 120 such that it can accommodate the larger diameter of the tubular shunt 114. This tapering minimizes the turbulence of blood flow. The wall thickness of the side tube 110 may also decrease at the distal end 120 of the side tube 110 to accommodate a match of bore size at the connection of the tubular shunt 114. One or more barbed ridges 122 are present on the outer wall of the side tube 110 to aid in the connection of the tubular shunt 114.

The cross section of FIG. 2 is also representative of the relative internal geometries of connector 104 (which is inserted into the vein). With respect to connector 104, the bore size of side tube 112 is approximately the same as straight base-tube 108 at the point of juncture 115 of the two tubes (108 and 112). To avoid turbulence of blood flow, the internal diameter of the side tube 112 gradually tapers outward from the juncture point to the distal end (shown in FIG. 1 being inserted inside shunt end 130). Again, the wall thickness of the side tube 112 may also decrease at its distal end to accommodate shunt connection. The distal end of tube 112 also includes barbed ridges.

Each anastomotic connector is secured with suture in such a way that the angled side conduits (e.g., conduits 110 and 112) protrude outward from the vein or artery. The outside diameters for each of portions 110 and 112 are sized relative to the inside diameters of tubular shunt portion 114 so that shunt 114 can be easily connected and secured to the side tube by suture. The non-porous property of the anastomotic connector prevents blood leakage and cellular in-growth into the walls of the connector. This unique non-porous property of the anastomotic connector is an advantage over the currently used porous-expanded PTFE arterial-venous shunt devices. Both leakage and cellular in-growth leading to intimal hyperplasia are attributed to the porous nature of the expanded PTFE material.

The angling of the side tubes 110 and 112 help regulate blood flow after installation. For each connector, the side tubes 110 and 112 are presented at a very narrow angle relative to the straight tube portions 106 and 108 respectively. This allows blood to flow through each connector with minimal turbulence. This is an advantage as turbulence is suspected of causing blood clotting and stenosis at the anastomosis. The narrow angle of juncture of the side tube to the straight tube of the anastomotic connector is about 20 degrees.

The bore size for each side tube (e.g., conduits 110 and 112) is sized such that blood flow through the shunt can be throttled downward to a desired amount. This is an advantage as it is known that blood flow must be limited or reduced between the artery and vein to prevent loss of adequate blood flow to the extremities distal to the AV Shunt. Excessive flow of blood though the shunt that reduces blood to the extremities is documented as a clinical problem called Steel Syndrome. The invention provides for a smooth blood flow transition to and from the larger bore tubular shunt such that turbulence is minimized, thus minimizing the potential for blood clotting.

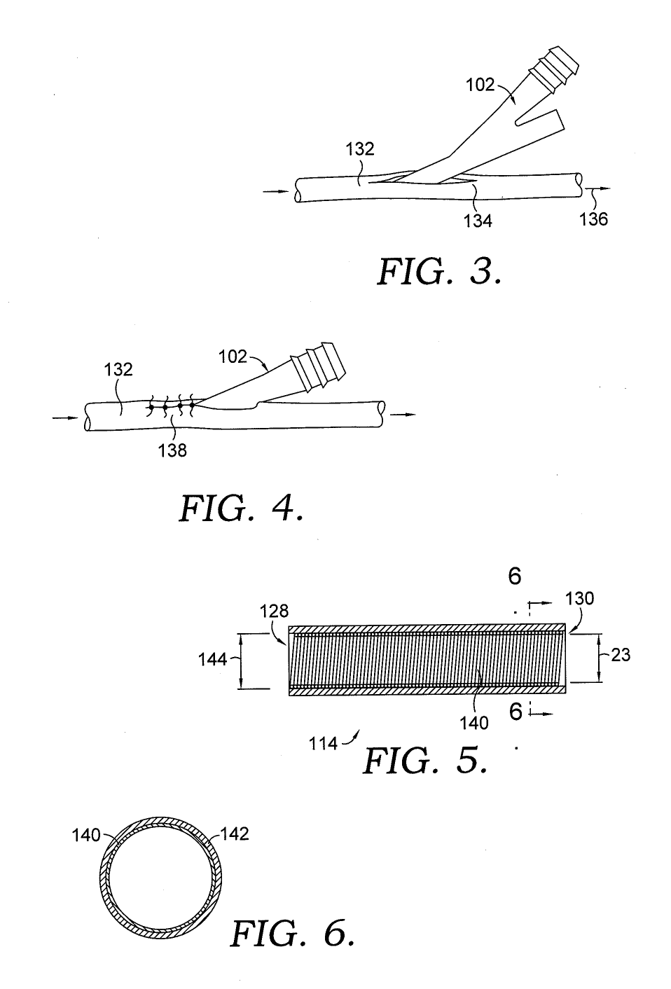

FIG. 3 is a three-dimensional drawing showing the anastomotic connector 102 being inserted into a typical artery 132. For a vein, the direction of blood flow would be reversed. A longitudinal slit 134 is made with a sharp instrument or scalpel and the straight base-tube portion of the connector 102 is placed into the artery 132. Care is taken that the correct orientation of the connector is maintained. The angle of the side tube 102 should face downstream relative to the blood flow 136 of the artery.

With respect to the blood-receiving vein, the angle of side tube 104 would be acute in the direction upstream relative to the blood flow of the vein, such that blood flow turbulence will be minimized when the diverted blood is received into the vein from the shunt.

FIG. 4 is a three-dimensional drawing showing the anastomotic connector 102 secured with sutures in the artery 132. In this drawing, several sutures 138 are used to secure the connector in-place to minimize leakage and assure minimal movement of the connector 102. Other suturing configurations can be used based on the surgeon's preference. Again, it should be understood that the connector 104 inserted into the vein would be sutured in an orientation relative to blood flow opposite that shown in FIG. 4.

FIG. 5 is a two-dimensional, longitudinal cross-sectional segmented view of a portion taken from the non-porous tubular shunt 114 of the AV shunt system 100. As can be seen, FIG. 5 does not show the shunt 114 in full length, but instead only depicts a portion of the full length for illustrative purposes. An outer silicone tube 142 is shown encasing an inner continuous spring or coil 140. The inside diameter of the silicone tube 142 is calibrated to the outer diameter of the spring or coil 140 such that a secure mechanical bond is maintained following placement of the spring or coil into the silicone tube during manufacture. The inside diameter 144 of the spring or coil 140 is selected based on the clinical needs of the patient.

In terms of materials, The non-porous tubular shunt 114 of the invention is constructed of an outer, blood compatible, non-porous silicone tube 142 (see FIGS. 5 and 6) and an inner, blood compatible, stainless steel spring or coil 140. The spring 140 is mechanically secured to the lumen of the silicone tube 142 via a press fit providing a composite assembly. The non-porous tubular shunt assembly results in a novel, resilient, minimally-leaking, non-porous tubular shunt 114 that allows for non-turbulent blood flow and easy access with needle or cannula puncture. Puncture with a needle is accomplished with minimal force through the silicone tube and between the coils of the spring. At removal of the needle, the spring coils snap back into their original positions. At removal of the needle, the silicone tubing, being elastic, retracts back to close the needle hole. The elastic nature of the silicone tube and the spring work in concert to close the needle hole, thereby minimizing leaks and mechanical damage to the tubular shunt.

FIG. 6 is a two-dimensional, transverse cross-sectional view of the tubular shunt portion of the AV shunt system. The outer silicone tube 142 is shown encasing the inner spring or coil 140. The wall thickness of the silicone tube is chosen to minimize the overall diameter of the tubular shunt while maintaining mechanical integrity and self-sealing properties. This view also shows how, in the embodiment depicted in FIG. 6, the coil 140 is located coaxially inside the outer self-sealing silicone tube 142. It should be noted, however, that in alternative embodiments the coil 140 could be located coaxially outside the tube. In yet further embodiments, two tubes could be utilized. In these embodiments (not shown), there would be a tube coaxially inside the coil 140 (not shown) and an additional tube located outside of the coil 140.

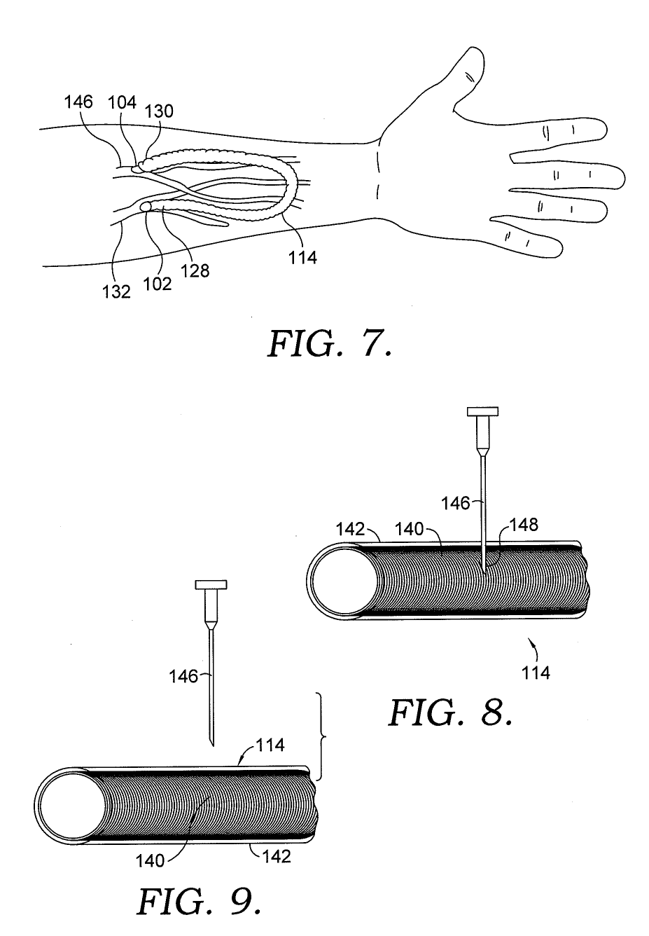

FIG. 7 is a three-dimensional schematic of the invention implanted in the forearm of a typical patient requiring hemodialysis. The anastomotic connectors 102 and 104 are placed into the patient's artery 132 and vein 146, respectively. First end 128 of tubular shunt 114 is shown attached to the angled conduit of connector 102 in artery 132, and second end 130 of shunt 114 is connected to the angled conduit of second connector 104 in vein 146. The AV shunt system is shown looped in the forearm presenting an access device for hemodialysis. The arterial-venous shunt system can also be implanted in various non-looped configurations based on the surgeon's preference and the clinical needs of the patient.

FIG. 8 is a close up three-dimensional view of the tubular shunt 114 being punctured by a needle 146. As a tip 148 of needle 146 is inserted through the silicone 142 wall of the tubular shunt 33, the elastic nature of the silicone tube ensures that the needle-hole size is minimized. The coils of the spring or coil 140, upon penetration of the needle, open allowing for easy passage there through so that hemodialysis can be performed.

Once hemodialysis has been completed, the needle is removed. FIG. 9 is a close up three-dimensional view of the tubular shunt 142 following removal of the needle 146. As the needle is pulled out, the coils of the spring 37 snap back into their original positions. This results in the automatic closure of the spring portion 140 of the tubular shunt. In addition, the outer silicone tube 142 snaps back into the needle hole due to the elastic nature of the silicone, providing self-sealing of the needle hole. The closing action of the spring coils and the elastic nature of the silicone work in concert to minimize leakage of the system.

### Example 1

### Needle-Hole Leakage Comparison of the Invention and Expanded PTFE (ePTFE) Graft

The performance of a shunt (e.g., like shunt 114 above) having a six mm bore size was compared to a conventional ePTFE Graft having a six mm bore size that is typically used for hemodialysis currently. Both devices were tested by an identical system that provides 100 mm static water pressure at ambient temperature. Needle puncture was accomplished with an 18 gauge hypodermic needle. Two test cases were chosen: a single puncture case and a multiple puncture case. Two (2) punctures were chosen for the multiple puncture case as this is the minimum number of punctures required per dialysis session for a typical patient on hemodialysis. Following removal of the needle, leakage through the resultant needle-hole was measured for a period of one minute. Results are presented as milliliters per minute.

TABLE I

| Sample | # Punctures | Leakage | #Punctures | Leakage |
|---|---|---|---|---|
| Shunt 114 | 1 | 0.5 ml/min | 2 | 1.5 ml/min |
| ePTFE | 1 | 94.0 ml/min | 2 | 208.0 ml/min |

As can be seen from Table I above, the leakage of the shunt disclosed in this application (e.g., shunt 114) was minimal for both puncture conditions. The leakage of the conventional ePTFE graft was about 100 to 200 times higher than shunt 114 in both puncture tests.

Significant leakage can result in a clinical problem called a seroma (leakage of serum creating a pocket of fluid under the skin) and can result in infection requiring removal of the ePTFE graft. To lessen this clinical risk, the standard practice in hemodialysis clinics is to wait for several weeks after implantation of the ePTFE graft to allow a thrombus coating to develop on the lumen of the graft. This coating helps lessen the leakage of the ePTFE graft to a more manageable level.

Using a shunt like that disclosed in this application avoids the need for this waiting period to be used in the hemodialysis clinic and the shunt (e.g., shunt 114) can be used immediately after implantation for potentially life-saving hemodialysis.

Those skilled in the art will recognize that the disclosed equipment is potentially usable for procedures other than hemodialysis, and that the disclosed invention should not be limited to any particular procedure or environment unless otherwise specified in the claims.

While preferred embodiments of the disclosed subject matter have been described, so as to enable one of skill in the art to practice the disclosed subject matter, the preceding description is intended to be exemplary only, and should not be used to limit the scope of the disclosure, which should be determined by reference to the claims pending at any relevant time.

We claim:

1. An arterial-venous shunt system comprising:
an arterial anastomotic connector for diverting blood into a shunt;
a venous anastomotic connector for returning the blood from the shunt to the body; and
the arterial and venous connectors being constructed of a biologically-compatible substantially nonporous material.

2. The system of claim 1 wherein the arterial anastomotic connector includes an insertable tubular portion and a diverting side conduit, the side conduit branching off from the tubular portion and extended such that blood is diverted at an angle which is acute relative to the direction of blood flow.

3. The system of claim 2 wherein the angle is about 20 degrees.

4. The system of claim 2 wherein the side conduit extends from a junction existing at about the middle of the insertable tubular portion.

5. The system of claim 1 wherein the venous anastomotic connector includes an insertable tubular portion and a receiving side conduit, the side conduit extending from the tubular portion at an angle such that blood will be received into a vein at an angle which is acute relative to the direction of blood flow.

6. The system of claim 5 wherein the angle is about 20 degrees.

7. The system of claim 5 wherein the side conduit extends from a junction existing at about the middle of the insertable tubular portion.

8. The system of claim 1 wherein at least one of the arterial and venous anastomotic connectors is made of substantially non-porous PTFE.

9. The system of claim 1 wherein the shunt is non-porous.

10. The system of claim 1 wherein the shunt is tubular.

11. The system of claim 1 wherein the shunt is self-sealing.

12. The system of claim 11 wherein the shunt includes an outer non-porous elastic tube and an inner tightly-wound coil.

13. The system of claim 12 wherein the tightly-wound coil is a metal spring which is bonded inside the non-porous elastic tube.

14. The system of claim 13 wherein the spring is made of stainless steel.

15. The system of claim 13 wherein the non-porous elastic tube is comprised of silicone.

16. The system of claim 1 wherein the shunt includes a tube in a coaxial relationship with a metal coil.

17. The system of claim 1 wherein the arterial anastomotic connector includes an insertable tubular portion having an inside diameter at an upstream that is substantially equal to an inside diameter of a selected artery.

18. The system of claim 1 wherein the venous anastomotic connector includes an insertable tubular portion having an inside diameter at an upstream that is substantially equal to an inside diameter of a selected vein.

19. A system for diverting a bodily fluid, the system comprising:
a diversion mechanism, said diversion mechanism being insertable into a human body for diverting a supply of fluid from a first bodily vessel into a second bodily vessel through a shunt, the shunt having a coaxial self-sealing arrangement, the coaxial self-sealing arrangement having an inner member and an outer member;
one of the inner and outer members being tubular and made of an elastic substance; and
the other of the inner and outer members being a tight coil, the coil being constructed to allow for the passage of a needle there through and then move back into place to close off an aperture created by the needle.

20. The system of claim 19 wherein the coil is located coaxially inside the tubular elastic member.

21. The system of claim 20 wherein the coil is a stainless steel spring and the tubular elastic member is made of silicone.

22. A method of artificially diverting blood from an artery into a vein, the method comprising:
surgically inserting a first substantially non-porous tubular PTFE member into an artery, the first PTFE member having an extraction conduit for diverting blood into a shunt;
surgically inserting a second substantially non-porous tubular PTFE member into a vein, the second PTFE member having an induction conduit; for receiving blood from the shunt; and
connecting the shunt between the extraction conduit and the induction conduit.

23. The method of claim 22 comprising:
forming a conduit by including a tightly-wound coil coaxially inside an elastic outer tube; and
using the conduit as the shunt in a surgical procedure.

24. The method of claim 22 wherein the extraction conduit is tapered away from a junction point with the first tubular PTFE member to correspond with a relatively larger inside diameter of the shunt, and the induction conduit is tapered such that it throttles down a return flow into the second tubular PTFE member.

25. A system for diverting blood from an artery into a vein, the system comprising:
a first substantially non-porous PTFE member having a tubular portion sized for insertion into an artery, the first PTFE member having an extraction conduit for diverting blood into a shunt;
a second substantially non-porous PTFE member having a tubular portion sized for insertion into a vein, the second PTFE member having an induction conduit for receiving blood from the shunt; and
the shunt being connectable between said extraction and induction conduits, the shunt an inner member and an outer member, said inner and outer members being coaxially related;
one of the inner and outer members being tubular and made of an elastic substance; and
the other of the inner and outer members being a tight coil, the coil being constructed to allow for the passage of a needle there through and then recoil to close off an aperture created by the needle.
